(19) United States
(12) Patent Application Publication
(10) Pub. No.: US 2017/0296381 A1
(43) Pub. Date: Oct. 19, 2017

(54) SPORTSWEAR COOLING SYSTEM

(71) Applicants: Paul Fox, White Plains, NY (US); Benjamin Wong, San Diego, CA (US)

(72) Inventors: Paul Fox, White Plains, NY (US); Benjamin Wong, San Diego, CA (US)

(21) Appl. No.: 15/488,376

(22) Filed: Apr. 14, 2017

Related U.S. Application Data
(60) Provisional application No. 62/322,402, filed on Apr. 14, 2016.

(51) Int. Cl. A61F 7/03; A41D 13/005; A41D 1/00; A41D 20/00; A61F 7/02; A61F 7/00

(52) U.S. Cl. CPC A61F 7/03; A41D 13/0053; A41D 13/0058; A41D 20/005; A41D 1/002; A41D 2400/62; A61F 2007/0095; A61F 2007/0219; A61F 2007/0238; A41D 2400/26

(57) ABSTRACT

A sportswear cooling system for enhancing physical activity for a wearer is provided which has a first compartment containing a cooling solution and a second compartment containing water. The first compartment is positioned within the second compartment and the walls of the first compartment are adapted to be ruptured so that when the cooling solution and water combine an endothermic reaction is produced calculated to cool a portion of the body upon application.

### CROSS-REFERENCE TO RELATED APPLICATION(S)

The present application claims priority to U.S. Provisional Patent Application No. 62/322,402 filed on Apr. 14, 2017, entitled “SPORTSWEAR COOLING SYSTEM” the entire disclosure of which is incorporated by reference herein.

### BACKGROUND OF THE INVENTION

### 1. Field of the Invention

The present invention relates to the field of wearable health and fitness sportswear. The invention provides a cooling function by targeting pulse point cooling areas and is designed to advance cooling of the body for faster recovery.

### 2. Description of Related Art

Overheating and body temperature fluctuations are common as a result of strenuous exercise. Overheating can increase fatigue and decrease productivity in athletic performance. When one experiences such feelings, it is common to bring a cool surface into contact with the body to absorb heat and to cool the overall temperature of the body. Devices that are useful for such purposes are known in the art as “cold packs”. For example, use of ammonium nitrate and water to initiate endothermic reactions for providing cooling effects have been widely discussed in the field of art.

Attempts have been made to utilize existing cold pack technology for different applications. However, existing cold packs are often cumbersome to use, and fail to stay securely in contact with a body part resulting in inefficient temperature reduction.

Vigorous exercise boosts one's body temperature and overall heat output production. As one's muscles warm up, blood circulating through the muscles is also heated and circulated throughout the entire body. This process produces an overall rise in core temperature. In comparison to other cooling products on the market that use passive cooling techniques, the present invention uses a small-targeted area cooling system that strives to maximize the cooling process at a core temperature level. This approach helps people stay cool, fight fatigue, and increases productivity during their workouts, hikes, runs, bike rides, and other strenuous activities.

Based on the foregoing, there is a need in the art for a device that places activated cooling capsules on targeted areas of the body, which allow the body to efficiently release energy, and accelerate the cooling of the overall body temperature during intense or extended periods of physical activity.

### BRIEF DESCRIPTION OF THE DRAWINGS

For a more complete understanding of the present invention, the objects and advantages thereof, reference is now made to the ensuing descriptions taken in connection with the accompanying drawings briefly described as follows.

FIG. 1 is a side elevation view of the wristband on an arm, in an embodiment of the invention.

FIG. 2 is a bottom plan view of the wristband on an arm, in an embodiment of the invention.

FIG. 3 is a bottom plan view of the compression sleeve on an arm, in an embodiment of the invention.

FIG. 4 is a side elevation view of the compression sleeve on an arm, in an embodiment of the invention.

FIG. 5 is a front cutaway view of the insulating cooling pocket, in an embodiment of the invention.

FIG. 6 is a side cutaway view of the insulating cooling pocket, without capsule inserted, in an embodiment of the invention.

FIG. 7 is a top cutaway view of the cooling capsule, in an embodiment of the invention.

FIG. 8 is a front cutaway view of the cooling capsule in an embodiment of the invention.

FIG. 9 is a side cutaway view of the cooling capsule in an embodiment of the invention.

FIG. 10 is a side cutaway view of unidirectional cooling design with capsule inserted in an embodiment of the invention.

FIG. 11 is a front cutaway view of the unidirectional cooling design cooling capsule inserted in an embodiment of the invention.

FIG. 12 is a bottom view of the compression sleeve with cooling capsule in an embodiment of the invention.

FIG. 13 is a top cutaway view of the compression sleeve with cooling capsule in an embodiment of the invention.

FIG. 14 is a bottom plane view of the compression sleeve with cooling capsule in an embodiment of the invention.

FIG. 15 is a bottom plane view of the compression sleeve with cooling capsule in an embodiment of the invention.

### DETAILED DESCRIPTION OF PREFERRED EMBODIMENTS

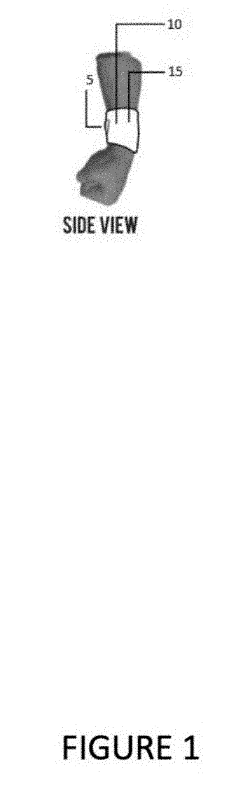

Preferred embodiments of the present invention and their advantages may be understood by referring to FIGS. 1-15 wherein like reference numerals refer to like elements.

With reference to FIGS. 1-15, the present invention pertains to athletic sportswear, compression sleeves and wristbands that contain pockets where an instant, replaceable cooling capsule 115 can be inserted. This invention also discloses a method for insertion and removal of cooling capsules from athletic sports gear designed to cool an athlete's core body temperature during physical activity. The pockets are located on the bottom side of the wrist (near where pulse rate may be measured), or near another targeted cooling area 25. The pockets are specially designed with an insulating material that lies over the exterior of the capsule 115 and a thin mesh material below. This design allows the endothermic process occurring within the capsule to maximize the cooling effects on the circulating blood. The instant cooling capsule is comprised of ammonium nitrate 50 and water 105 in two or more separate compartments. Once a small force of pressure activates the cooling capsule 115, the water 105 compartment 110 will break, flowing into the ammonium nitrate chamber. The energy released by the solvation of the ammonium ions and nitrate ions is less than the energy absorbed in breaking up the ammonium nitrate ionic lattice and the attractions between water 105 molecules. This combination of the two elements will create an endothermic reaction creating a cooling effect. The strategic placement and location of the cooling capsules on one's body allows for cooled blood to re-circulate and lower the body's overall temperature in a quick and efficient manner. These cooling capsules may be placed on other body parts such as the forehead, temples, ankles, and neck.

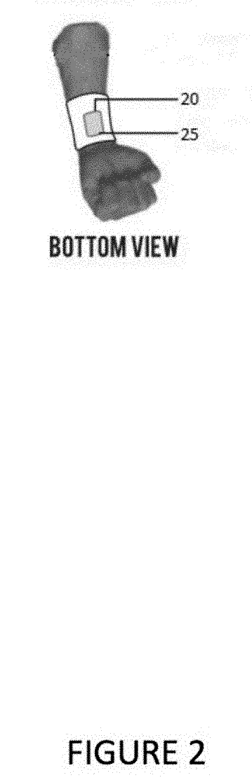

With reference to FIG. 1 and FIG. 2, a wristband 95 with a targeted cooling area 25, which can be worn while exercising, is shown. In one embodiment, the wristband is shown with an elastic insulating pocket 5. The wristband is shaped in a manner that allows for the maximum surface area of the cooling capsule 115 to be placed flush against the user's wrist. The wristband can be designed with Spandex™ elasticity to form fit around the wearer's arm to further maximize surface area contact with the capsule 115. In some embodiments, the wristband is stretched over the hand and onto the wrist. In another embodiment, the wristband can be attached around the forearm with use of a clasp, VELCRO™, or other fastening device(s). Further, stitching is minimally visible to allow for a tight fit around the forearm of the user to keep the wristband in place during periods of intense and rapid physical motion. In an embodiment, the ultraviolet protection factor (UPF) 10 is interwoven into the wristband to provide the user protection from harmful UV rays while enjoying outdoor activities. In FIG. 2, a bottom planar view with targeted cooling area 25 and elastic opening 20 is shown. In some embodiments, the wristband is composed of a UPF 10 material and also composed of hydravent moisture wicking technology 15 to pull moisture away from the skin. In some embodiments the hydravent technology 15 is composed of an inner layer made of hydrophobic synthetic fiber bonded to an outer layer. An optimized blend of hydrophobic and hydrophilic fibers will speed up drying time. In other embodiments, the fibers can be composed of polyester, spandex, or LYCRA™ material to help wick moisture. In an embodiment, the wristband or compression sleeve includes a visual display that conveys information about cooling time, such as how much has transpired, or how much longer cooling will proceed.

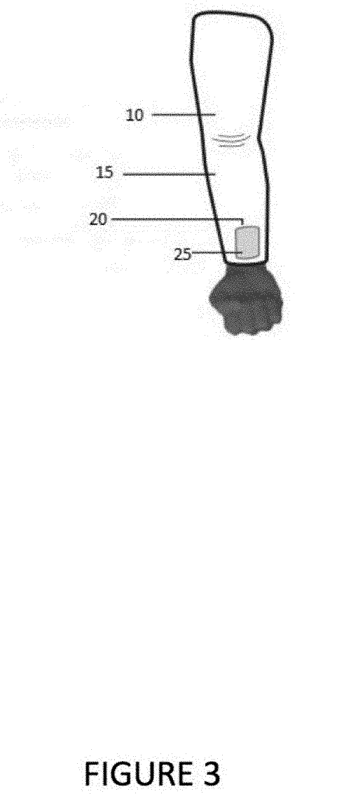

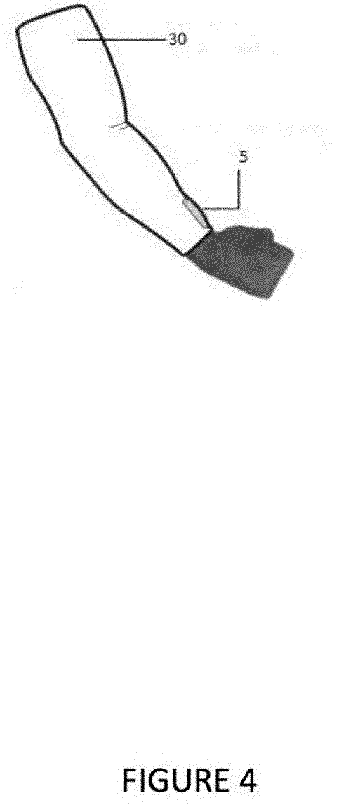

With reference to FIG. 3 and FIG. 4, an embodiment of a compression sleeve shows the form fitting fabric material 30 and elastic insulating pocket 5. In an embodiment, the lower third of the compression sleeve contains a reinforced elastic layer to provide a secure fit closer to the user's wrist. The compression sleeve will go from the user's wrist up to the shoulder region. In some embodiments, the compression sleeve can further be reinforced and tightened through use of a VELCRO™ strap or other fastener. In an embodiment, the fabric material is DRI-FIT™. The contour and placement of the pocket provides for a secure and snug fit of the cooling capsule against the user's wrist. In one embodiment, the stitching allows for a comfortable fit and provides a mesh pocket base 35 to reduce the weight of the pocket. The ultraviolet protection factor 10 (UPF) is present along with the hydravent moisture wicking material 15 across the entirety of the compression sleeve to provide maximal protection against UV rays.

Outdoor physical activity subjects people to UV rays, often for extended periods of time when hiking, running, or performing other physical activities. The targeted cooling area 25 is located inside of the elastic insulating pocket 5. Said elastic insulating pocket 5 provides an elastic opening 20 for the cooling capsule 115 to be inserted inside the targeted cooling area 25. In an embodiment, the compression sleeve is form fitting fabric material 30 and contains an elastic opening 20 with reflective material near the targeted cooling area 25, in which the replaceable capsules can be inserted. The opening allows for efficient thermal energy transfer and for the placement of noninvasive capsules to increase comfort, mobility, and range of motion.

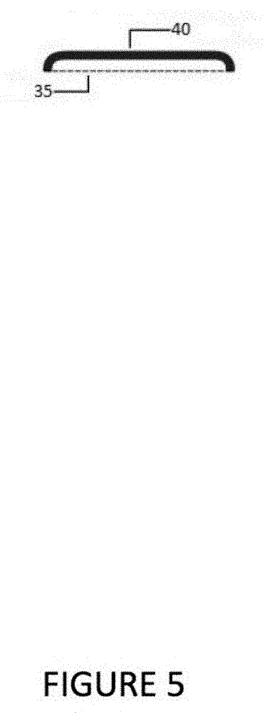

In an embodiment, with reference to FIG. 5, the targeted cooling exterior pocket 40 is white in color in order to inhibit heat absorption and to increase cooling capsule 115 effectiveness duration. In another embodiment, the targeted area can be any reflective color to function to alert drivers if the user is running at night or hiking in the dark. The targeted cooling area 25 may have a reflective outer layer, which can reflect UV rays and energy from the sun.

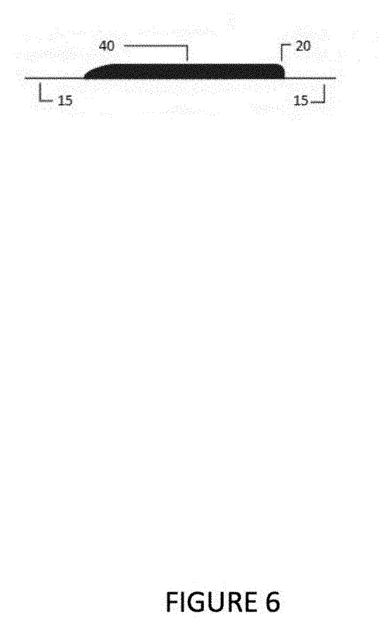

With reference to FIG. 5 and FIG. 6, an embodiment provides for an elastic insulating exterior pocket 40 which may be stretched apart from the mesh pocket base 35 so the cooling capsule 115 can sit in between. The elastic insulating exterior pocket 40 stretches to make it easier for the user to remove and insert cooling capsules. The elastic insulating exterior pocket 40 provides insulation via its composition of synthetic materials, providing resistance to heat transfer from the natural surroundings. In an embodiment, a radiant barrier helps prevent the exterior pocket from exposure to mold or mildew which can commonly be found in active sports wear found in the market after excessive use. The insulating exterior pocket also reduces capsule 115 movement during periods of intense physical activity. The elastic insulating exterior pocket 40 is located in a functional location for mobility. Additionally, the cooling pocket has dense elastic moisture wicking exterior to further insulate a replaceable cooling capsule 115. The mesh pocket base 35 provides a lightweight support for holding the cooling capsule 115 in place and allows for a larger surface area in which the thin endothermic transfer layer 80 can make contact with the user as opposed to traditional cotton, nylon, or other materials. In an embodiment, there is a gel base layer affixed to the mesh pocket base 35 which the capsule 115 may rest on. The gel base layer can slow the cooling process of the cooling capsule 115 by providing a barrier between the cooling capsule and heat from the body or environment.

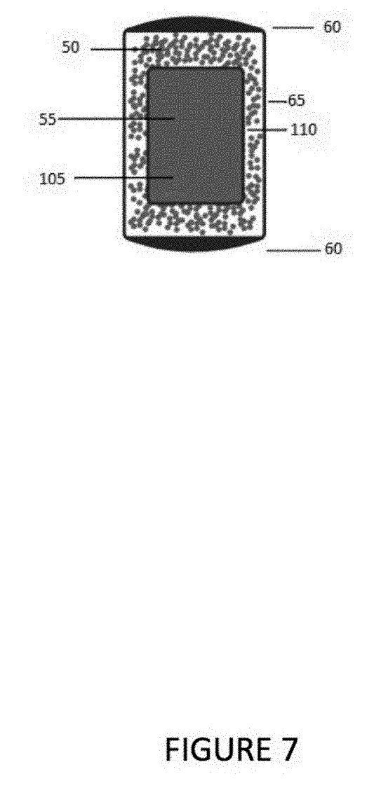

With reference to FIG. 7, a cooling capsule 115 is shown. In an embodiment, the cooling solution 50 within a cooling capsule 115 can be composed of urea, ammonium nitrate, and/or water 105. In one embodiment, the cooling solution 50 within the cooling capsule 115 is comprised of ammonium nitrate 50 and water 105 in two separate compartments. In an embodiment, a liquid solution is added to the cooling packet that changes color as the reaction takes place over a period of 20 minutes. Food dye can be added to the ammonium nitrate solution, and another dye is added to the water 105. After the two solutions in separate compartment have been mixed, the user can see how much time has elapsed since the cooling capsule 115 has been activated based on the color change that takes place gradually over time.

In another embodiment, cooling agents besides urea, ammonium nitrate, and water may be used. Further, a capsule 115 which becomes cold through a different endothermic reaction, or where a barrier dissolved in water may provide the cooling sensation to the user.

In another embodiment, the capsule 115 has a digital communication system, which can connect to a smart watch or phone to alert the user that their body temperature is getting too high, or that the cooling reaction has finished. In an embodiment, the quick pull tab 60 is made of a plastic sheath which protrudes from the cooling capsule 115. This allows a non-aqueous piece of the cooling capsule 115 to be visible to the user and not covered by the elastic insulating pocket 5. In an embodiment, the quick pull tab 60 can be manufactured using a 3D printing durable plastic shell, which can be placed on top of the capsule 115 that can protect the capsules from impact. This visible protrusion allows the user to easily remove and replace cooling capsules when using the compression sleeves or wristbands for extended periods of time. In an embodiment, the quick pull tab 60 serves to remove the capsule 115 from the targeted cooling pocket and is capable of creating a large seal area within the targeted cooling area 25 during use to prevent gas emission or breakage during physical activity.

In an embodiment, the capsule 115 is designed with insulation walls 75 and air cavities to prevent environmental penetration. In an embodiment, the capsule 115 interior wall uses a thin plastic wall to direct energy transfer to the body and away from the environment. The sleeve-cooling pocket has a thin mesh material to allow flow of energy between the body and the capsule 115. In an embodiment, the elastic cooling pocket opening closes, to protect the cooling capsule 115 from outside exposure to natural elements.

In another embodiment, the cooling solution 50 is comprised of a gel substrate impregnated with a polymer gel and an antimicrobial agent. The gel substrate may have a PVA/PVP or PVA/PVA blend base.

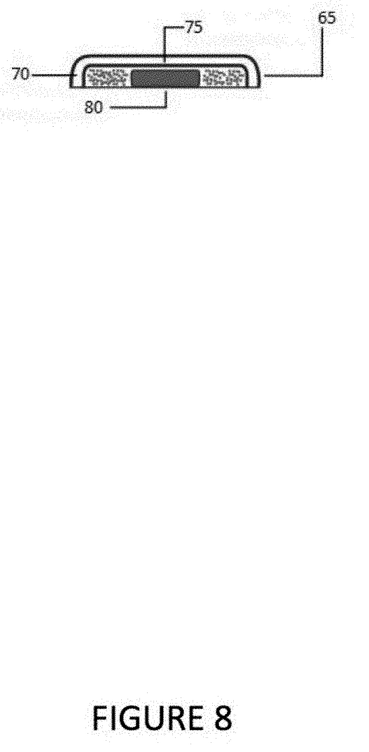

With reference to FIG. 8, a replaceable cooling capsule 115 is shown. In an embodiment, the capsule 115 features a unidirectional thermal energy transfer architecture in which the insulating layer is placed on top of the capsule 115 to reduce energy loss to the environment. On the bottom of the capsule 115, a thin plastic base will allow for thermal transfer. The endothermic transfer layer 80 is flexible, and promotes a unidirectional thermal energy transfer from the user's skin. Opposite the insulation walls 75, heat diffuses through a more porous, and flexible surface on the bottom side of the capsule 115. In an embodiment, the endothermic transfer layer 80 is covered with a thin color film to prompt the user to place that side of the capsule 115 in towards his body. In an embodiment, the capsule 115 contains multiple layers of walls on the topside to further insulate the cooling solution 50. Multiple paned layers on the outer walls create insulation. Embodiments in which the capsule 115 has multiple walls enable the capsule 115 to maximize reaction time and cooling benefits. The endothermic transfer layer 80 consists of a lower density material than the other capsule 115 layered walls on the opposite side to provide greater insulation for the cooling solution 50.

In another embodiment, the capsule 115 has a quick activation internal chamber to separate the two or more cooling solutions 50 located in the capsule 115. Said quick activation chamber 55 provides stability in the capsule 115 for interstate transport to retail stores, but is easy enough to break apart without having to shake vigorously. Once the chamber has been broken, the unidirectional thermal energy transfer will provide for lower energy loss to the environment while maintaining flexibility and comfort. In an embodiment, the capsules last for 15-20 minutes in duration.

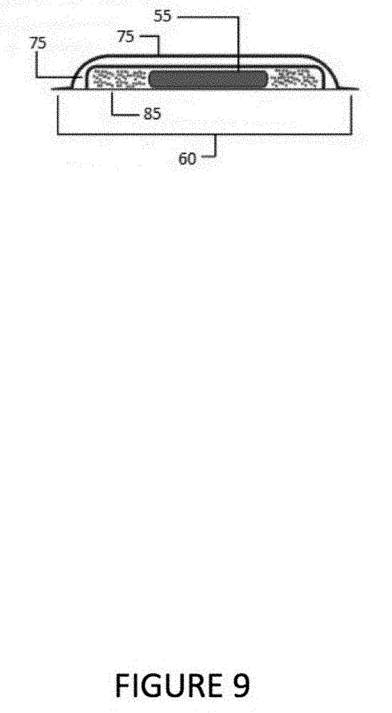

With reference to FIG. 9, a replaceable cooling capsule 115 is shown. A feature of the device is that the capsules will be replaceable, enabling the cooling process to be repeated as frequently and as many times as necessary during physical or strenuous activity. When a cooling capsule 115 has been activated, cooled blood will circulate throughout the body, cooling the body at the core level. In an embodiment the cooling capsules shall contain a capillary system in which two aqueous solutions are unable to obtain high flow velocity. In this embodiment, the cooling capsules are able to last for longer than 20 minutes in duration. In an embodiment, the capsule 115 exterior wall (top) uses a denser material for insulation. The flexible base layer 85 of the capsule 115 allows for more of the capsule 115 to adhere to the user's body or to help avoid becoming twisted, contorted, or perpendicular to the user's wrist during strenuous physical activity.

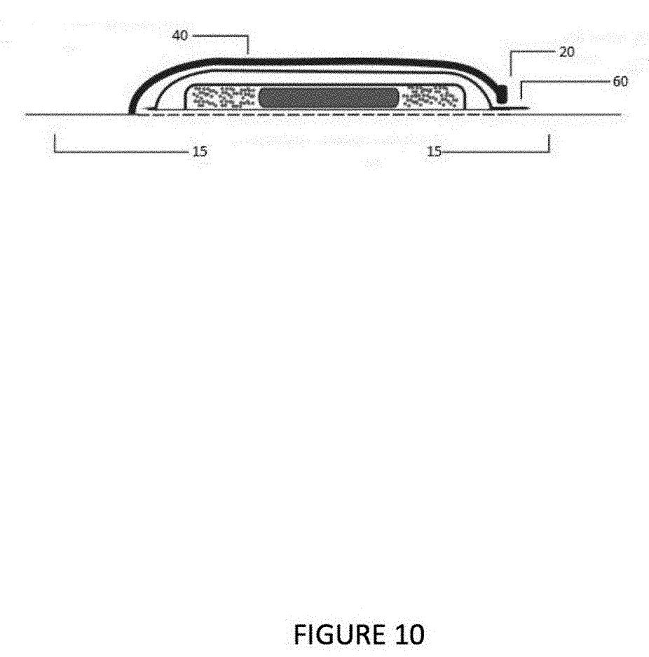

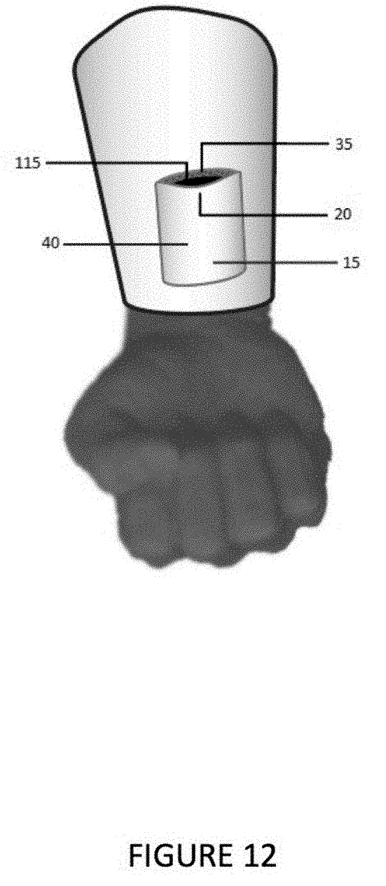

With reference to FIG. 10 and FIG. 12, in an embodiment the hydravent moisture wicking material 15 is adjacent to both sides of the elastic insulating exterior pocket 40. In an embodiment, the mesh pocket base 35 sits flush against the user's skin and adjacent to the hydravent moisture wicking material 15. In another embodiment, the hydravent moisture wicking material 15 is also lining the mesh pocket base 35 of the elastic insulating pocket 5 to increase moisture removal capability. The elastic opening 20 can be stretched to place cooling capsule 115 in place.

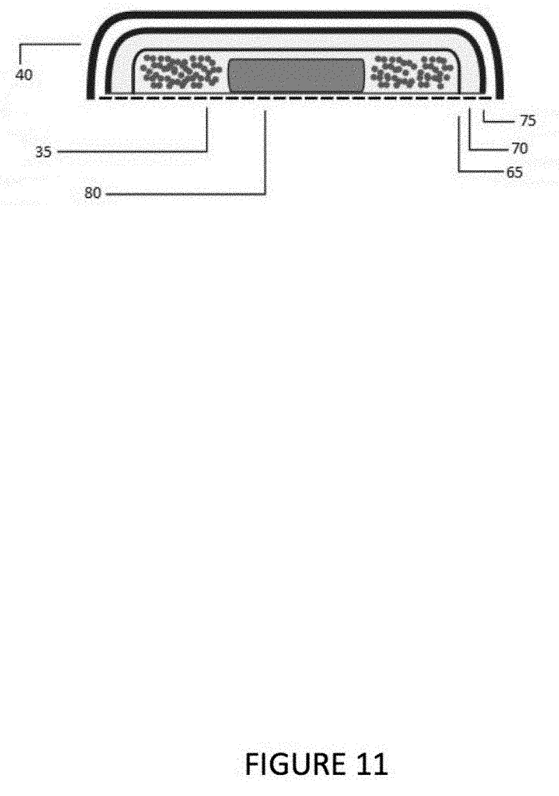

With reference to FIG. 11, in an embodiment the capsule 115 aids in the unidirectional cooling design by using a capsule interior wall to create an air cavity 70 to prevent environmental penetration and to trap energy loss from diffusing outside of the elastic insulating pocket 5.

With reference to FIG. 7, a process is disclosed for a method of removal and replacement of gel cooling capsules from athletic sportswear. A process for removal of ammonium nitrate and water 105 solutions within cooling capsules affixed to athletic sportswear comprising the steps of gripping said capsule 115 by a quick pull tab 60 which is permanently adjoined to the cooling capsule 115.

The process set forth wherein said quick pull tab 60 is a sheath of plastic with ribbed texture for easier removal of a cooling capsule 115 from an exterior elastic insulating pocket 40 affixed to athletic sportswear. In an embodiment, the shape of the quick pull tab 60 will be curved for comfort and also to lay flat or adjacent to the cooling pocket opening. The elastic opening 20 will close on top of the quick pull tab 60 to secure the capsule 115 in place.

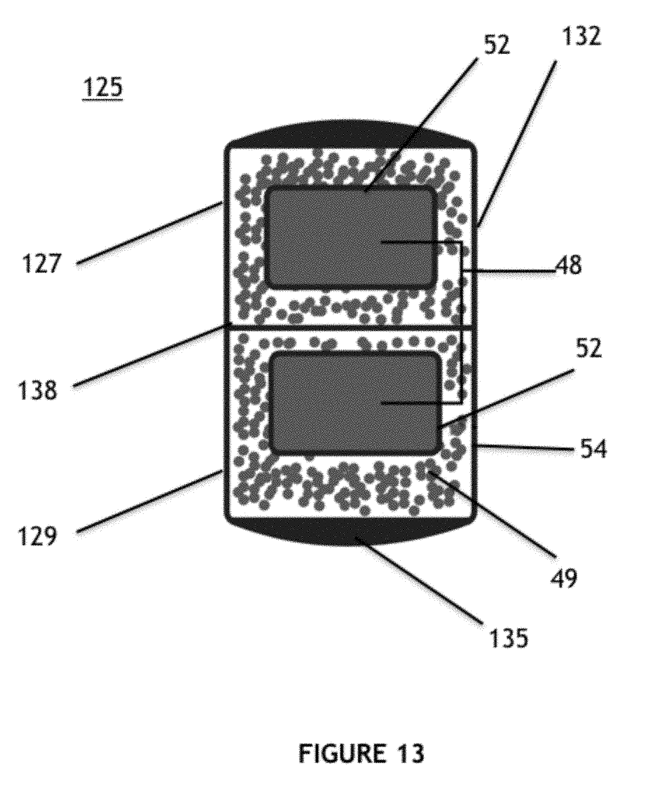

With reference to FIGS. 13-15, a dual chamber a cooling capsule 125 is shown. The outside of the capsule 125 is formed of a flexible protective wall 132, with an optional internal chamber separation wall 138 separating the capsule 125 into two portions, an upper portion 127 and a lower portion 129. The double portions allow the user to activate the capsules in two parts, thereby doubling the cooling duration. For instance, it may be the case that with a single portion capsule, the initial cooling may cool down the skin within, first instance, the first 10 minutes without a user feeling any additional cooling even though the capsule is still below 70° F. By introducing two activations, through two capsule portions, the capsules' effects on the user may be amplified.

In an embodiment, the cooling solution 48 within a cooling capsule 125 can contain urea, ammonium nitrate, and/or water. In one embodiment, the cooling solution 48 within the cooling capsule 125 is comprised of ammonium nitrate within a first compartment(s) 52 (in this embodiment there are two) and water 49 within a second compartment 54 (in this case coinciding with the walls of the capsule 125), these constituents in two separate compartments. There may be a plurality of compartments 52, 54 (more than two) for ammonium nitrate and water. In an embodiment, a liquid solution is added to the cooling packet that changes color as the reaction takes place over a period of 20 minutes or so, a similar time to the cooling effect of the capsule 125, such that the user may gauge the length of cooling time elapsed or remaining. Food dye can be added to the ammonium nitrate solution, and/or another dye may be added to the water 54. After the two solutions in separate compartment have been mixed, the user can see how much time has elapsed since the cooling capsule 125 has been activated based on the color change that takes place gradually over time. The ammonium nitrate 48 and water 49 can have reversed positions, wherein the cooling solution 48 is found in the second compartment 54 and the water is found in the first compartment 52.

The dual chamber capsule 125 has a pull tab 135. Pull Tab 135 is a tool used for the wearer to easily remove the capsule from a pocket.

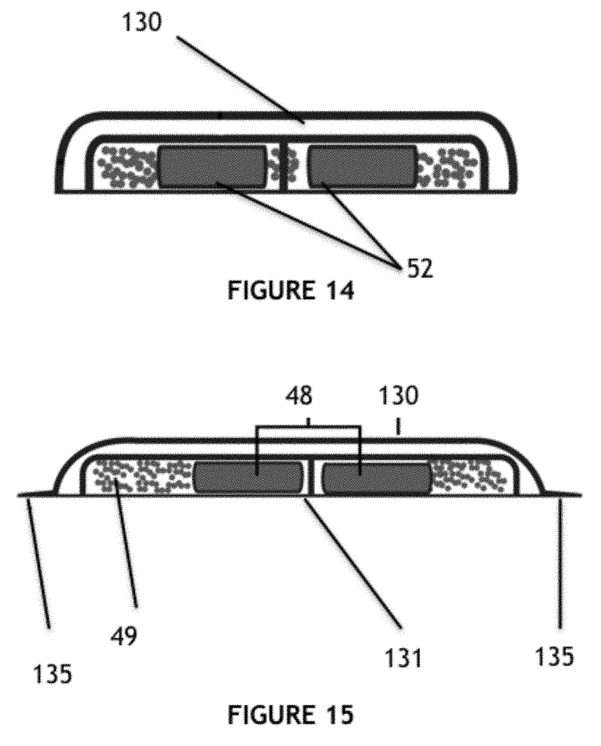

With reference to FIG. 14, and in an embodiment, the capsule 125 has a top insulated wall 130 that is insulated with a double wall or otherwise, containing either insulative material such as flexible foam, or air between the layers, and may have a reflective metallized layer (not shown) on the interior to assist with reflecting the cold of the capsule 125 back into the capsule. It also has an uninsulated wall 131 on the bottom, facing the skin such that the cold is efficiently transferred to the user. In an embodiment, this bottom wall 131 is a single layer, and it may consist of a metallized surface to transfer heat energy more efficiently from the user's skin to the cold capsule 125.

The benefit of the dual chamber capsule 125 is incremental introduction of the solution for a longer cooling duration, and better mixing, obviating the need for shaking of the capsule 125. The capsule has an instant cooling upon pressure activation. A unidirectional cooling design has a double wall insulation design at the top, to prevent energy loss to environment, and has a thin thermal transfer layer for efficient cooling, on the bottom where the capsule 125 is in contact with the user's skin.

Preferred embodiments of the present invention and their advantages may be understood by referring to FIGS. 1-15, wherein like reference numerals refer to like elements.

The invention is described using words and phrases for illustrative purposes only. Terms and phrases are intended to cover plural forms and grammatical variations and are not limiting the scope and implementation of the invention.

The invention has been described herein using specific embodiments for the purposes of illustration only. It will be readily apparent to one of ordinary skill in the art, however, that the principles of the invention can be embodied in other ways. Therefore, the invention should not be regarded as being limited in scope to the specific embodiments disclosed herein.

I claim:

1. A capsule for cooling a wearer, comprising:
a. a first compartment containing a cooling solution;
b. a second compartment containing water, wherein the first compartment is positioned within the second compartment;
c. wherein the walls of the first compartment are adapted to be ruptured and when the cooling solution and water combine an endothermic reaction is produced.

2. The capsule of claim 1 wherein the cooling solution is ammonium nitrate.

3. The capsule of claim 1 wherein the second compartment has an exterior wall and an contact wall, wherein the exterior wall comprises a double wall for insulation, and the interior wall is a single wall adapted to be placed against a user and to facilitate heat transfer.

4. The capsule of claim 1 further comprising a pull tab removal of the capsule.

5. Temperature cooling apparel comprising:
a. a flexible capsule having a first compartment containing a first substance;
b. a second compartment, within the capsule, abutting the first compartment, and separated therefrom by the second compartment containing a second sub stance;
c. an elastic, insulating pocket extending over a portion of the capsule; and
d. a compression fabric for holding the capsule at a body application site in connection with the capsule producing an exothermic reaction as a result of a ruptured wall between the first and second compartments.

6. The temperature cooling apparel as recited in claim 5 wherein the apparel comprises a wristband.

7. The temperature cooling apparel as recited in claim 6 which further includes a fastening device for securing the wristband to a wrist.

8. The temperature cooling apparel as recited in claim 6 wherein the wristband is made of material consisting of hydrophobic synthetic fiber, polyester and a combination thereof.

9. The temperature cooling apparel as recited in claim 5 wherein the first substance is water and the second substance is a cooling solution selected from the group consisting of urea, ammonium nitrate and a combination thereof.

10. The temperature cooling apparel as recited in claim 9 wherein the second substance includes dye.

11. The temperature cooling apparel as recited in claim 5 further comprising a digital communication system for use with a smart phone or a watch operable to send a body temperature alert in connection with predetermined the body temperature changes.

12. The temperature cooling apparel as recited in claim 5 wherein said second substance comprises a gel substrate.

13. The temperature cooling apparel as recited in claim 12 wherein the gel substrate is impregnated with polymer gel and an antimicrobial agent.

14. The temperature cooling apparel as recited in claim 13 wherein the gel substrate has a PVA/PVP of PVA/PVA blend base.

15. The temperature cooling apparel as recited in claim 5 which further comprises
a. a second flexible capsule having a first compartment containing the first substance; and
b. a second compartment, within the second flexible capsule, abutting the first compartment of the second flexible capsule, and separated therefrom by the second compartment, of the second flexible capsule, containing a second sub stance.

16. The temperature cooling apparel as recited in claim 5 further comprising a pull tab connected to the flexible capsule.

17. The temperature cooling apparel as recited in claim 15 further comprising a pull tab connected to the second flexible capsule.

18. The temperature cooling apparel as recited in claim 15 further comprising a hydravent moisture wicking material extending over the elastic insulating pocket.

19. The temperature cooling apparel as recited in claim 18 further comprising a reflective outer layer over a portion of the elastic insulting pocket.

20. The temperature cooling apparel as recited in claim 19 wherein the reflective outer layer is capable of reflecting ultra-violet rays.
